(19) United States
(12) Patent Application Publication
(10) Pub. No.: US 2014/0319750 A1
(43) Pub. Date: Oct. 30, 2014

(54) GRIPPING SYSTEM FOR STRAIGHTENING A BALLOON CATHETER DURING FLUTING

(71) Applicant: Interface Associates, Inc., Laguna Niguel, CA (US)

(72) Inventors: David E. Yanes, Laguna Beach, CA (US); Eric Mabry, Trabuco Canyon, CA (US); Malik Sumnani, Irvine, CA (US)

(73) Assignee: Interface Associates, Inc., Laguna Niguel, CA (US)

(21) Appl. No.: 14/265,967

(22) Filed: Apr. 30, 2014

Related U.S. Application Data
(60) Provisional application No. 61/817,802, filed on Apr. 30, 2013.

(51) Int. Cl. A61M 25/10

(52) U.S. Cl. CPC A61M 25/1038; A61M 25/1029
USPC 269/86; 29/559

(57) ABSTRACT

An apparatus for gripping a balloon catheter during folding (i.e., fluting) is provided. The apparatus includes a base unit, a back gripper and a front gripper. The front gripper is configured to straighten an inner lumen of the balloon catheter. A method for gripping a balloon catheter during fluting is also provided. The method can include the steps of straightening an inner lumen of the balloon catheter and aligning the inner lumen with a central axis when straightened. The method further can include the steps of providing a plurality of arms coupled to the base unit and arranged in a pattern about a central axis, translating the plurality of arms in a radial direction toward and away from the central axis, fluting the balloon catheter with a plurality of blades coupled to the plurality of arms.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57, including but not limited to U.S. Prov. Pat. App. No. 61/817,802 filed on Apr. 30, 2013.

### BACKGROUND

1. Field

The present invention generally relates to balloon catheters. More particularly, the present invention relates to devices and methods used to grip a balloon catheter and/or guide wire during fluting.

2. Description of the Related Art

A balloon catheter generally comprises an inflatable balloon that is mounted along a distal end of an elongate catheter body (e.g., a shaft). Balloon catheters are used by physicians in a wide variety of therapeutic procedures. In one common use, a folded balloon catheter is advanced through a blood vessel to a region that has become occluded by atherosclerotic plaque. The balloon is inflated to dilate the occluded region and thereby improve the flow of blood through the vessel. In another common use, an expandable stent is provided along the exterior of the balloon. The balloon is advanced to the treatment site and is then inflated to deploy the stent. The balloon is then deflated and the balloon catheter is withdrawn from the patient. The expanded stent remains in the blood vessel to provide support to the vessel wall.

Balloon catheters are typically formed of a very thin, yet strong material. During manufacture, a balloon catheter is folded at a number of locations along its longitudinal axis. After the balloon is folded in a variety of locations, the folds are wrapped around the catheter to reduce the balloon to a constrained condition having a very small diameter. In the case wherein the balloon is used to deploy a stent, the stent is crimped onto the balloon after the folding step. The balloon is advanced through the blood vessel to a treatment site while in the constrained condition. The balloon is inflated by directing a fluid through a lumen in the catheter to pressurize the balloon. During inflation, the balloon unfolds, rather than stretches. Embodiments of the present invention enable a balloon catheter to radially expand in a very predictable manner.

### SUMMARY

Embodiments of the present invention disclosed herein provide improved devices and methods for gripping a balloon catheter during fluting. The embodiments facilitate the manufacture of balloon catheters while minimizing costs and providing a balloon catheter that expands in a reliable and predictable manner. Further, the embodiments provide a modular unit that is capable of gripping balloon catheters of a variety of different sizes and shapes.

In some embodiments, a desirable feature for balloon catheters is a small profile so as to be able to use the smallest introducer possible. In addition to the design of the catheter, e.g., proximal neck outer diameter and balloon wall thickness, the fluting and wrapping of the balloon around the shaft can play a significant role. The inner lumen traversing the length of the balloon catheter may not be well centered during the fluting process. This offset between the center axis of the fluter system and the inner lumen of the balloon catheter can lead to uneven flutes, a poor wrap and/or catheter damage.

The length of the inner lumen relative to the length of the balloon catheter typically varies due to differences in design and different compliances during inflation. The unsupported inner lumen of the balloon catheter can become bowed or adopt a serpentine shape within the balloon. For balloon catheters designed for use with larger diameter guide wires, for example 0.018″ or larger diameter guide wires (e.g., 0.035″ diameter guide wires), the bowing may not be as significant since a guide wire or stylet of similar size is typically used during fluting. The larger diameter guide wires, in some cases, provide sufficient stiffness so as to straighten out the inner lumen of the balloon catheter. However, for balloon catheters designed for use with smaller diameter guide wires, for example 0.014″ or smaller diameter guide wires (e.g., 0.010″ diameter guide wires, or no more than about 0.016″, 0.015″, 0.014″, 0.013″, 0.012″, 0.011″, 0.010″, 0.009″, 0.008″ or less in diameter guide wires), the stiffness of the guide wire can be insufficient to provide enough support. The smaller diameter guide wires, in some cases, do not provide sufficient stiffness so as to straighten out the inner lumen and/or the balloon catheter. The issue of bowing is further complicated by the ever increasing length of balloons, with some manufacturers offering 200-300 mm length balloons or more, or at least 150 mm, 175 mm, 200 mm, 225 mm, 250 mm, 275 mm, 300 mm, 325 mm, 350 mm, 375 mm, 400 mm, or more. With these longer lengths, the extent of bowing of the inner lumen of the balloon catheter increases significantly.

In one embodiment, an apparatus for gripping a balloon catheter during folding (i.e., fluting) is provided. The apparatus includes a base unit, a first, e.g., back gripper and a second, e.g., front gripper. The back gripper is removably coupled to a first portion of a balloon catheter. The front gripper is removably coupled to a second portion of the balloon catheter. The front gripper is configured to straighten an inner lumen of the balloon catheter. The inner lumen aligns with a central axis when straightened. A plurality of arms is coupled to the base unit and arranged in a pattern about the central axis. The plurality of arms can translate in a radial direction toward and away from the central axis. A plurality of blades can be coupled to the plurality of arms such that the plurality of blades can engage the balloon catheter during fluting.

A method for gripping a balloon catheter during fluting is also provided. Such method can include the steps of providing a device having a base unit, a back gripper and a front gripper; removably coupling the back gripper to a first portion of a balloon catheter; removably coupling the front gripper to a second portion of the balloon catheter. The method further can include the steps of straightening an inner lumen of the balloon catheter; aligning the inner lumen with a central axis when straightened. The method further can include the steps of providing a plurality of arms coupled to the base unit and arranged in a pattern about the central axis; translating the plurality of arms in a radial direction toward and away from the central axis; fluting the balloon catheter with a plurality of blades coupled to the plurality of arms.

### BRIEF DESCRIPTION OF THE DRAWINGS

These and other features, aspects, and advantages will now be described in connection with some embodiments of the invention in reference to the accompanying drawings. The illustrated embodiments, however, are merely examples and are not intended to limit the invention. The drawings include the following fourteen figures.

FIG. 1 is a perspective view of a balloon catheter having a straight inner lumen, according to some embodiments of the invention.

FIG. 2 is a cross-sectional view of the balloon catheter of FIG. 1.

FIG. 3 is a cross-sectional view of the balloon catheter of FIG. 1 with flutes.

FIG. 4 is a perspective view of a balloon catheter having a bowed inner lumen, according to some embodiments of the invention.

FIG. 5 is a cross-sectional view of the balloon catheter of FIG. 4.

FIG. 6 is a cross-sectional view of the balloon catheter of FIG. 4 with flutes.

FIG. 7A is a front elevational view of a fluter system, according to some embodiments of the invention, showing a plurality of arms and a plurality of blades disposed about a central axis such that each of the plurality of arms and each of the plurality of blades is capable of translating toward the central axis and away from the central axis; FIG. 7B is another view of the fluter system of FIG. 7A.

FIG. 8 is a front view of a back gripper, according to some embodiments of the invention.

FIG. 9 is a perspective view of a back gripper.

FIG. 10 is a side view of a front gripper, according to some embodiments of the invention.

FIG. 11 is a side view of a gripping system, according to some embodiments of the invention.

FIG. 12 is a side view of a gripping system with a balloon catheter.

### DETAILED DESCRIPTION

Disclosed herein are systems and methods to advantageously straightening the balloon catheter and/or inner lumen during the fluting process. In some embodiments, disclosed is a first, e.g., back gripper that centers and grabs the one or more supporting guide wires. On another portion of the balloon catheter, a second, e.g., front gripper supporting the catheter shaft is centered, such as on the same axis as the back gripper. With the shaft centered, the operator can provide tension, straightening the guide wire, by, for example, either pulling directly on the guide wire or grasping it through the catheter shaft. Once there is sufficient tension, the fluting process can be initiated and the inner lumen can be centered within the flutes.

In some embodiments, a modification to this approach is to add a tensioner, e.g., as a function of the front gripper, the back gripper, or a separate component. With this approach the guide wire can be grabbed by the front gripper, directly or indirectly, and tension applied by the tensioner. Any of the grippers, such as the front gripper and/or the back gripper, can be pneumatic, mechanical and/or electrical, as well as the tensioner.

Once the fluting is completed, the balloon can be kept under vacuum to maintain the flutes and centering of the inner lumen. The balloon catheter is transferred to a wrapping station or manually wrapped (use of vacuum can often be used for fluters and wrappers).

The balloon catheter gripping system described herein is a cost effective tool that can be particularly well suited for balloon catheter manufacturing. These improved devices can substantially speed-up, simplify, and improve the balloon folding and wrapping processes. The balloon catheter gripping system described herein may be used with a wide variety of balloon catheter sizes and shapes. The balloon catheter gripping system can be provided as separate units. Each gripper and/or tensioner can be controlled by a control unit (e.g., pneumatic, mechanical, electrical, and/or magnetic).

The front gripper and the back gripper each can comprise a mechanism to grip the balloon catheter and/or a guide wire extended through the balloon catheter. The tensioner e.g., movement of the front gripper or pulling by the operator, can straighten the balloon catheter and/or the guide wire. The movement of the blades wraps the folds around a catheter shaft so as to provide a relatively small diameter.

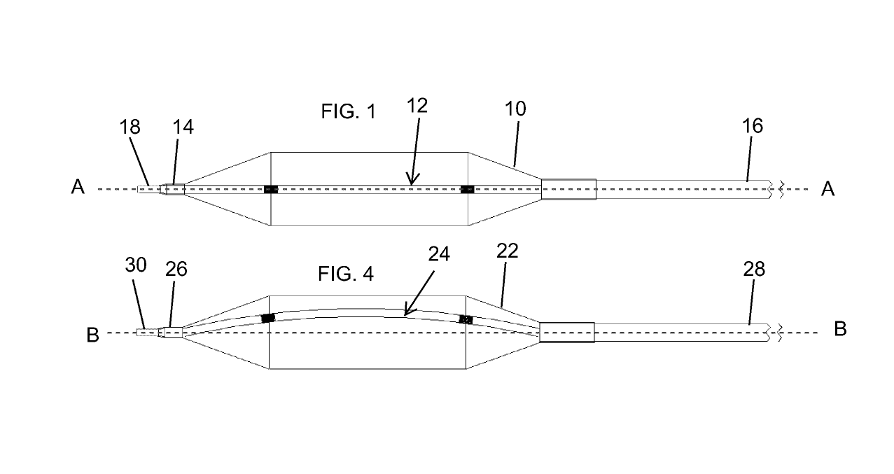

As shown in FIG. 1, a balloon catheter 10 can have a straight inner lumen 12. In some embodiments, the straight inner lumen 12 is supported so as to be straight or substantially straight within the balloon catheter 10. For balloon catheters designed for use with larger diameter guide wires, for example 0.018″ or larger diameter guide wires (e.g., 0.035″ diameter guide wires), the straight inner lumen 12 may be straightened during fluting. The larger diameter guide wire or stylet of similar size is typically used during fluting to straighten the balloon catheter 10. The larger diameter guide wires can often provide sufficient stiffness so as to straighten out the straight inner lumen 12 of the balloon catheter 10.

The straight inner lumen 12 can be straight, substantially straight, partially straight, and/or other linear configurations. The straight inner lumen 12 extends through at least a portion of the balloon catheter 10. In some embodiments, the straight inner lumen 12 can extend from a proximal end 14 of the balloon catheter 10 to a distal end 16 of the balloon catheter 10. In other embodiments, the straight inner lumen 12 extends through a portion of the length of the balloon catheter 10.

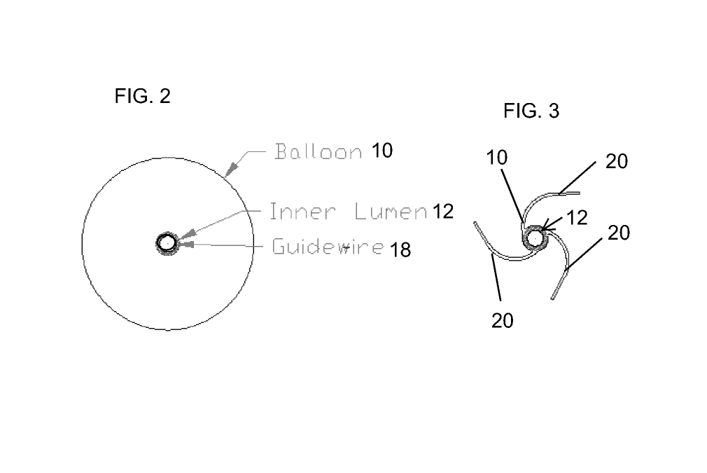

FIG. 2 shows a cross-sectional view of the balloon catheter 10. In some embodiments, the straight inner lumen 12 is supported so as to be coaxial within the balloon catheter 10. In other words, the straight inner lumen 12 extends along the longitudinal axis A- A of the balloon catheter 10, see FIG. 1. A guide wire 18 can extend through the straight inner lumen 12 in order to support the straight inner lumen 12 during fluting. The guide wire 18 can be coaxial with the balloon catheter 10 and/or the straight inner lumen 12. The straight inner lumen 12 is centered within the balloon catheter 10. The balloon catheter 10 having a straight inner lumen 12 can provide acceptable flutes or pleats.

FIG. 3 shows a cross-sectional view of the balloon catheter 10. The balloon catheter 10 has been folded to create flutes 20. FIG. 3 shows three flutes, but other embodiments are contemplated. The folding process can create even flutes 20. These even flutes 20 will typically fold well. The straight inner lumen 12 may linear along the longitudinal axis A-A of the balloon catheter 10, see FIG. 1. The flutes 20 may be regular along the length of the balloon catheter 10.

As shown in FIG. 4, a balloon catheter 22 can have a bowed inner lumen 24. The bowed inner lumen 24 can be curved, substantially curved, partially curved, bowed, substantially bowed, partially bowed, serpentine, substantially serpentine, partially serpentine and/or other non-linear configurations. The bowed inner lumen 24 can be caused by the balloon catheter 22 being unsupported.

For balloon catheters designed for use with smaller diameter guide wires, for example 0.014″ or smaller diameter guide wires (e.g., 0.010″ diameter guide wires), the stiffness of the guide wire can be insufficient to provide enough support during fluting. The smaller diameter guide wires do not provide sufficient stiffness so as to straighten out the inner lumen and the balloon catheter during fluting. With longer lengths of balloon catheters, the extent of bowing of the inner lumen of the balloon catheter increases significantly. The bowed inner lumen 24 is not centered within the balloon catheter 22. The balloon catheter 22 having a bowed inner lumen 24 can potentially create uneven and lumpy flutes that may not fold well.

The bowed inner lumen 24 extends through at least a portion of the balloon catheter 22. In some embodiments, the bowed inner lumen 24 can extend from a proximal end 26 of the balloon catheter 22 to a distal end 28 of the balloon catheter 22. In other embodiments, the bowed inner lumen 24 extends through a portion of the length of the balloon catheter 22.

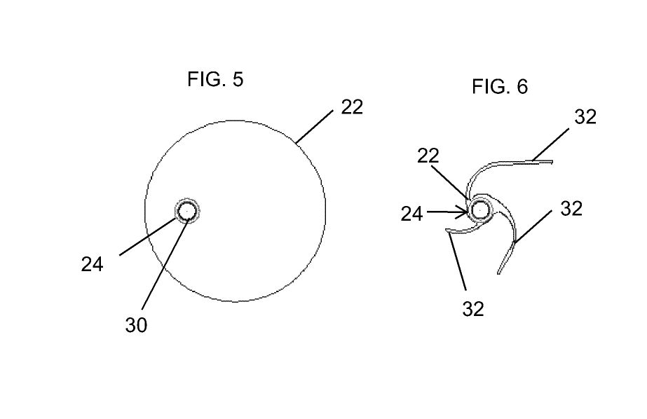

FIG. 5 shows a cross-sectional view of the balloon catheter 22. In some embodiments, the bowed inner lumen 24 is unsupported so as to be not coaxial within the balloon catheter 22. In other words, the bowed inner lumen 24 is offset from the longitudinal axis B-B of the balloon catheter 22. A guide wire 30 can extend through the bowed inner lumen 24. The guide wire 30 can be coaxial with the bowed inner lumen 24. The guide wire 30 can be insufficient to straighten the bowed inner lumen 24 during fluting. The guide wire 30 can be offset from the longitudinal axis B-B of the balloon catheter 22, see FIG. 4. In some embodiments, the gripping system can increase the straight-line distance between two ends of the bowed inner lumen 24 moving from a first bowed configuration to a second linear configuration after gripping. In some embodiments, the straight-line distance between two ends of the bowed inner lumen 24 can increase by at least about 2%, 3%, 4%, 5%, 6%, 7%, 8%, 9%, 10%, 12%, 15%, 20%, 25%, 30%, 40%, 50% or more from the linear relative to the bowed configuration, or various ranges between any of the aforementioned values, such as from about 2% to about 20% for example.

FIG. 6 shows a cross-sectional view of the balloon catheter 22. The balloon catheter 22 has been folded to create flutes 32. FIG. 6 shows three flutes 32, but other embodiments, having one, two, four, five, six, or more flutes are contemplated. The balloon catheter 22 having a bowed inner lumen 24 can provide uneven and lumpy flutes that may not fold well. As shown, the folding process created uneven flutes 32. These uneven flutes 32 may not fold well. The bowed inner lumen 24 may not be consistently offset from the longitudinal axis B-B of the balloon catheter 22, see FIG. 4. The flutes 32 may be irregular along the length of the balloon catheter 22.

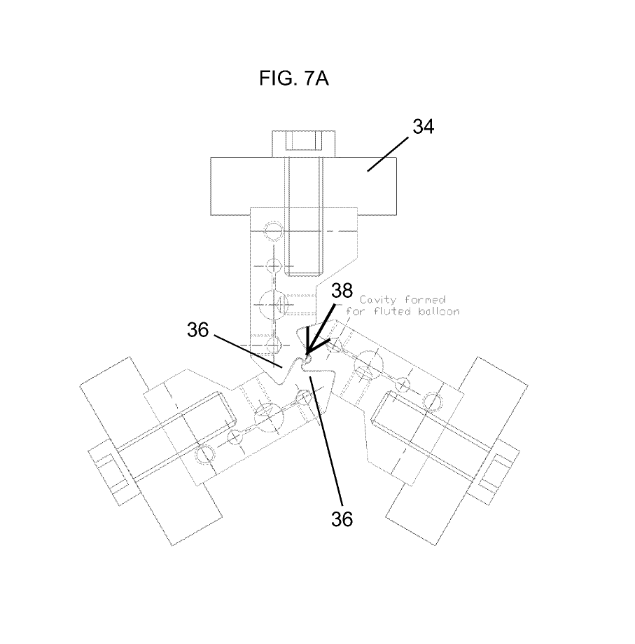

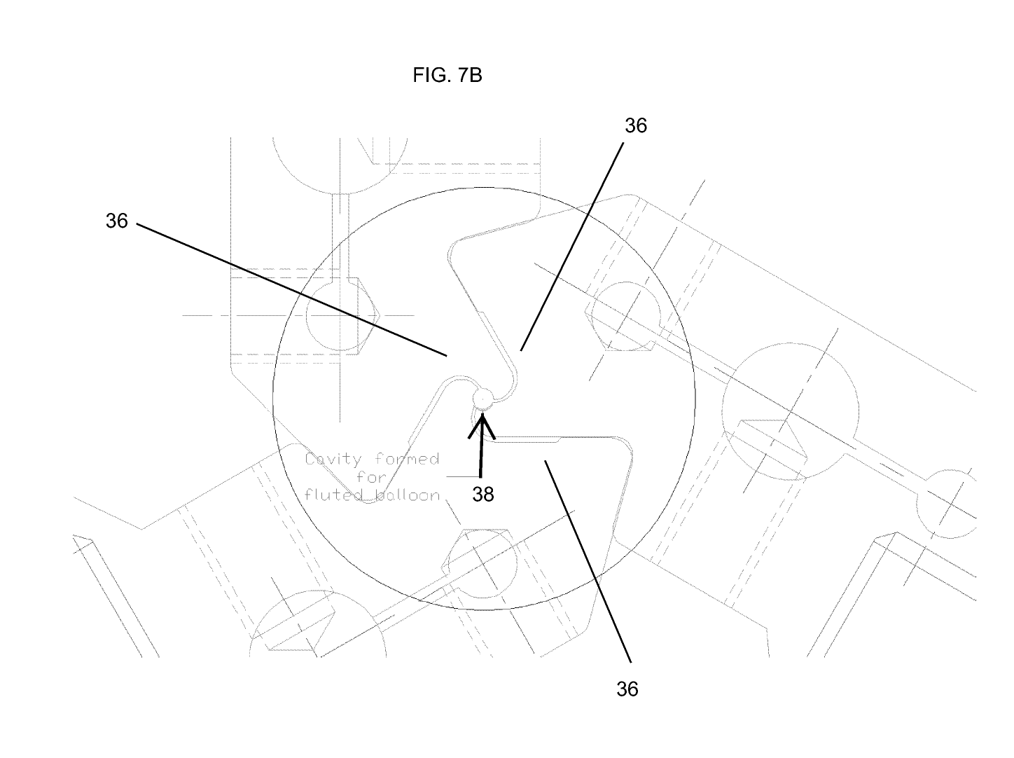

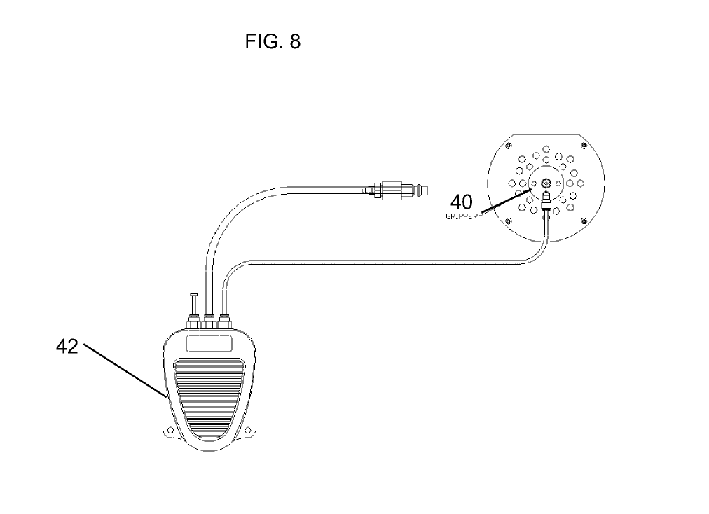

FIGS. 7A-7B illustrates a three blade fluter system 34 having a plurality of blades 36. A cavity 38 is defined by the convergence of the plurality of blades 36. The cavity 38 is configured to receive a balloon catheter, such as the balloon catheter 10, 22. The cavity 38 can have a longitudinal axis C-C, see FIG. 11. If the inner lumen 12, 24 of the balloon catheter 10, 22 is not centered within the cavity 38 as the plurality of blades 36 come together, then the flutes could be uneven and/or the catheter could be damaged. In other words, the inner lumen 12, 24 of the balloon catheter 10, 22 should align with the longitudinal axis C-C of the cavity. In some embodiments, the inner lumen 12, 24 of the balloon catheter 10, 24 should be coaxial with longitudinal axis C-C of the cavity. The methods and devices for balloon catheter folding are further described in commonly owned, U.S. Pat. No. 7,762,804, the disclosure of which are hereby incorporated by reference in their entirety and should be considered a part of this specification.

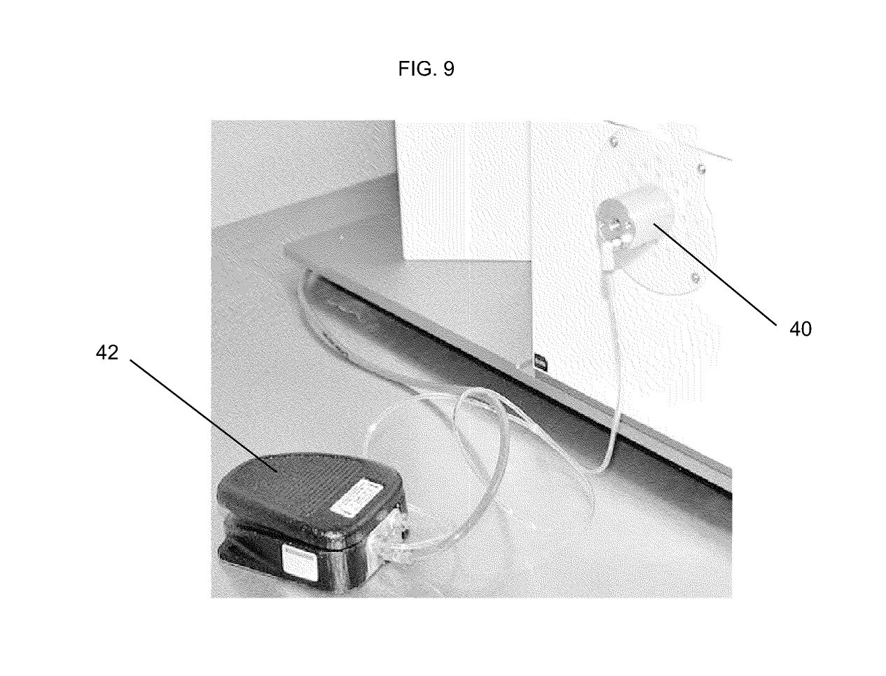

FIG. 8 shows a front view of a back gripper 40. FIG. 9 shows a perspective view of a back gripper 40. The back gripper 40 can be configured to grasp the proximal end 14, 26 of the balloon catheter 10, 22. The back gripper 40 can be configured to grasp the balloon catheter 10, 22 along a length of the balloon catheter 10, 22. In some embodiments, the back gripper 40 can be configured to grasp a portion of the balloon catheter 10, 22.

In some embodiments, the back gripper 40 centers and grabs the one or more supporting guide wires 18, 30. The back gripper 40 can be configured to grasp one end of the guide wire 18, 30. The back gripper 40 can be configured to grasp the proximal end 14, 26 of the balloon catheter 10, 22 and one end of the guide wire 18, 30. The back gripper 40 can be configured to grasp the balloon catheter 10, 22 and/or the guide wire 18, 30 disposed therewithin. The back gripper 40 can be located on a first end of the fluter system 34.

The back gripper 40 can be controlled by a control unit (e.g., pneumatic, mechanical, electrical, and/or magnetic). FIGS. 8-9 show a pneumatic back gripper. In some embodiments, the back gripper 40 is coupled to a foot pedal 42 or another control to actuate the back gripper 40. In some embodiments, the foot pedal 42 can be coupled to a fluid source. The foot pedal 42 can be used to actuate the back gripper 40 between an open configuration wherein the balloon catheter 10, 22 and/or the guide wire 18, 30 is not grasped and a closed configuration wherein the balloon catheter 10, 22 and/or the guide wire 18, 30 is grasped.

In order to align the back gripper 40, the mounts can be made with a small amount of float. A pin (not shown) with a diameter generally the same as or similar to the balloon catheter 10, 22 can be placed in the fluter system 34. The plurality of blades 36 can be closed around the pin. The back gripper 44 can be centered mechanically on the pin. Then, the mounts can be tightened.

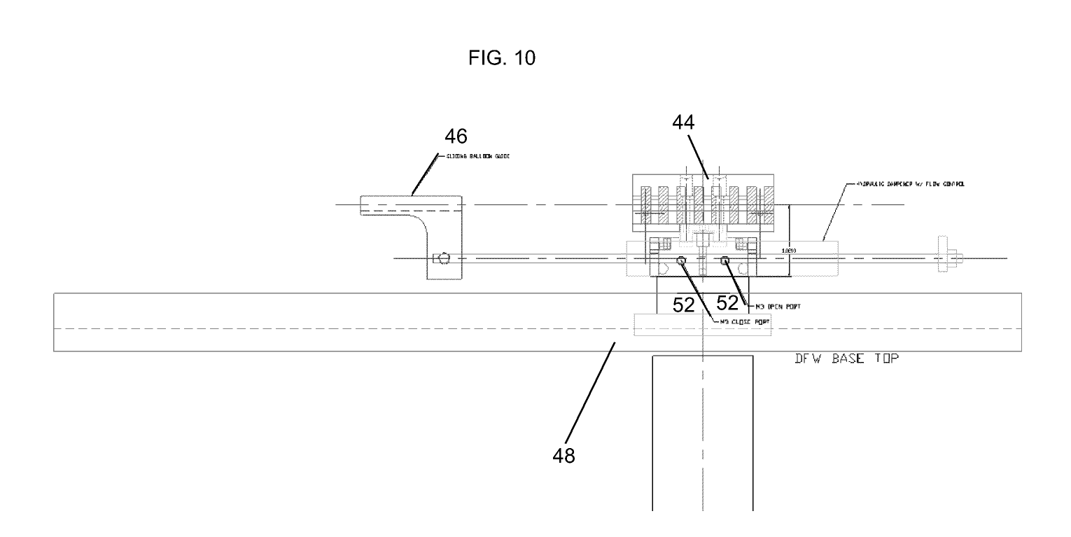

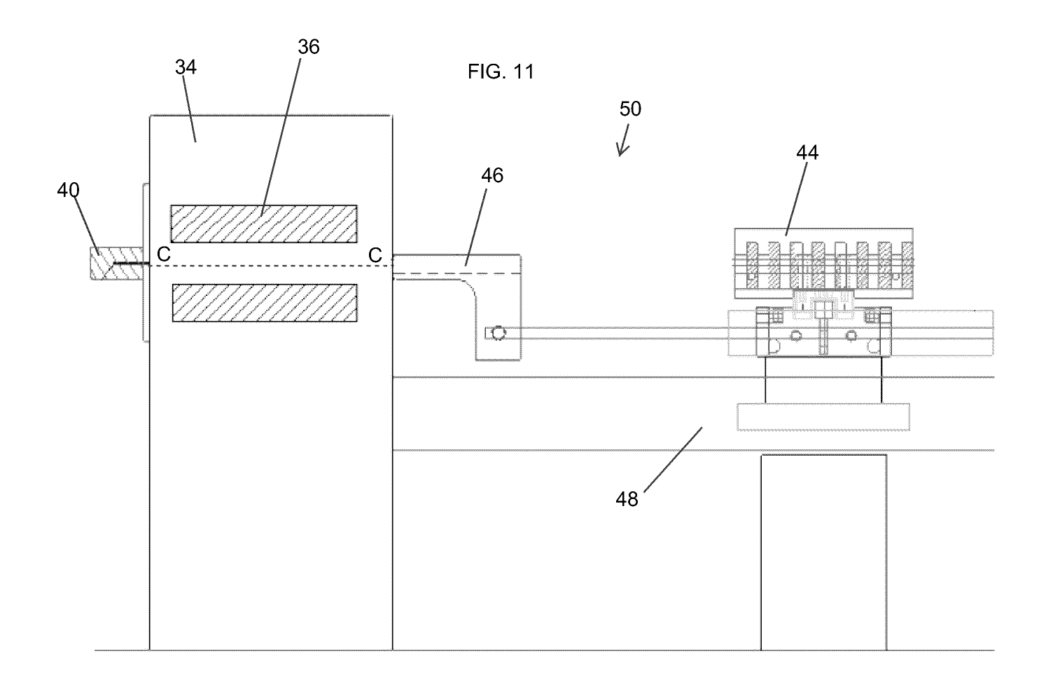

FIG. 10 shows a side view of a front gripper 44. The front gripper 44 can be configured to grasp the distal end 16, 28 of the balloon catheter 10, 22. The front gripper 44 can be configured to grasp the balloon catheter 10, 22 along a length of the balloon catheter 10, 22. In some embodiments, the front gripper 44 can be configured to grasp a mid- portion of the balloon catheter 10, 22. In some embodiments, the front gripper 44 can be configured to grasp a portion of the balloon catheter 10, 22. In some embodiments, the front gripper 44 can be configured to grasp the shaft of the balloon catheter 10, 22. The larger area of the front gripper 44 distributes the force of the front gripper 44.

In some embodiments, the front gripper 44 centers and grabs the one or more supporting guide wires 18, 30. The front gripper 44 can be configured to grasp one end of the guide wire 18, 30. The front gripper 44 can be configured to grasp the distal end 16, 28 of the balloon catheter 10, 22 and one end of the guide wire 18, 30. The front gripper 44 can be configured to grasp the balloon catheter 10, 22 and/or the guide wire 18, 30 disposed therewithin. The front gripper 44 can be located near the second end of the fluter system 34

The front gripper 44 can be coupled to a base 48. The base 48 can be coupled to a fluter system, such as fluter system 34. The front gripper 44 can be controlled by a control unit (pneumatic, mechanical, electrical, magnetic). In some embodiments, the base 48 includes ports 52 to accept fluid. The ports 52 can be used to actuate the front gripper 44 between an open configuration wherein the balloon catheter 10, 22 and/or the guide wire 18, 30 is not grasped and a closed configuration wherein the balloon catheter 10, 22 and/or the guide wire 18, 30 is grasped.

In some embodiments, a front gripper 44 can be coupled to a sliding balloon guide 46. The sliding balloon guide 46 can be configured to slide along a length of the balloon catheter 10, 22. In some embodiments, the sliding balloon guide 46 can be configured to slide along a mid-portion of the balloon catheter 10, 22. The sliding balloon guide 46 can be spaced from the front gripper 44. In some embodiments, the sliding balloon guide 46 is disposed between the back gripper 40 and the front gripper 44. In some embodiments, the sliding balloon guide 46 is configured to provide radial support for the balloon catheter 10, 22. In some embodiments, the sliding balloon guide 46 is configured to facilitate the straightening of the balloon catheter 10, 22 and/or guidewire 18, 30.

The sliding balloon guide 46 can be located on a second end of the fluter system 34. The sliding balloon guide 46 can be centered on the same axis as the back gripper 40, the front gripper 44, the longitudinal axis A-A, the longitudinal axis B-B, and/or the longitudinal axis C-C. In one embodiment, the sliding balloon guide 46 is a rail centered on the same axis as the front gripper 44 grasping the balloon catheter 10, 22.

With the shaft of the balloon catheter 10, 22 centered, the operator can provide tension. The operator can apply tension by any suitable means of applying tension known in the art. For instance, the operator can pull directly on the guide wire or pull the guide wire indirectly through pulling the shaft of the balloon catheter 10, 22.

In some embodiments, the gripper system includes a tensioner, e.g., a function added to the front gripper 44, a sliding balloon guide 46, the back gripper 40 or a separate component. With this approach the guide wire 18, 30 can be grabbed by the gripper system, directly or indirectly, and tension applied. For instance, a pneumatic spring can apply tension. For instance, the front gripper 44 can be configured to apply tension to the balloon catheter 10, 22. For instance, the front gripper 44 can translate linearly along the base 48 to apply tension. The front gripper 44 can apply tension after the front gripper 44 grasps the balloon catheter 10, 22. For instance, the sliding balloon guide 46 can be configured to apply tension to the balloon catheter 10, 22. For instance, the sliding balloon guide 46 can translate linearly along the base 48 to apply tension. The sliding balloon guide 46 can apply tension after the front gripper 44 grasps the balloon catheter 10, 22.

Any of the grippers, such as the back gripper 40 and the front gripper 44, can be pneumatic, mechanical and/or electrical. The tensioner, such as the tensioning provided by the front gripper 44, can be pneumatic, mechanical and/or electrical.

Once there is sufficient tension, the fluting process can be initiated. Due to the tension, the inner lumen of the balloon catheter 10, 22 can be centered within the flutes. Once the fluting is completed, the balloon catheter 10, 22 can be kept under vacuum to maintain the flutes and centering of the inner lumen and transferred to a wrapping station or manually wrapped (use of vacuum is typical for fluters and wrappers).

FIG. 11 shows a side view of a gripping system 50. The gripping system 50 can include the back gripper 40. The back gripper 40 can be configured to grasp the balloon catheter 10, 22 and/or the guide wire 18, 30 disposed therewithin. In some embodiments, the back gripper 40 can be coupled to the fluter system, such as fluter system 34. The fluter system 34 can include the plurality of blades 36. The cavity 38 defined by the convergence of the plurality of blades 36. The cavity 38 is configured to receive a balloon catheter, such as the balloon catheter 10, 24. The cavity 38 can have a longitudinal axis C-C.

The gripping system 50 can include the front gripper 44. The front gripper 44 can be configured to grasp the balloon catheter 10, 22 and/or the guide wire 18, 30 disposed therewithin. The front gripper 44 can be coupled to a sliding balloon guide 46. In some embodiments, the sliding balloon guide 46 can be coupled to the fluter system 34. The front gripper 44 can be coupled to a base 48. In some embodiments, the base 48 can be coupled to the fluter system 34.

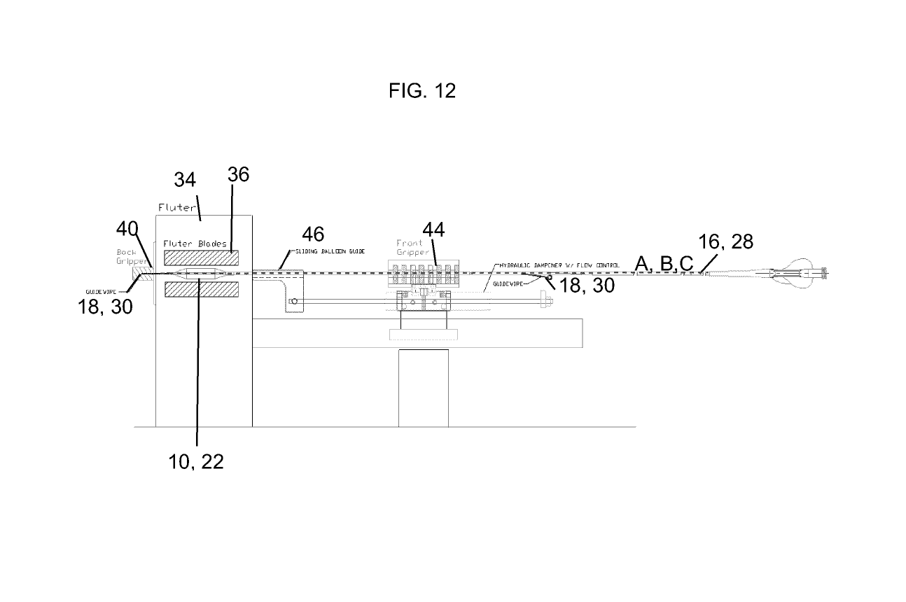

In some methods, a balloon catheter 22 can have a bowed inner lumen 24 is placed within the cavity 38, as shown in FIG. 12. The cavity 38 has longitudinal axis C-C. The bowed inner lumen 24 is offset from the longitudinal axis B-B of the balloon catheter 22. The bowed inner lumen 24 does not align with the longitudinal axis C-C. In some embodiments, the longitudinal axis B-B of the balloon catheter 22 aligns with the longitudinal axis C-C. If the blades 36 were to close on the balloon catheter 22 in this configuration, then the flutes may be uneven, see FIG. 6.

In some methods, the back gripper 40 grasps the balloon catheter 22 and/or the guide wire 30 disposed therewithin. The front gripper 44 grasps the balloon catheter 22 and/or the guide wire 30 disposed therewithin. In some embodiments, the sliding balloon guide 46 slides along the length of the balloon catheter 22. In some embodiments, the sliding balloon guide 46 is disposed between the back gripper 40 and the front gripper 44.

The tensioner can be configured to apply tension to the balloon catheter 10, 22 and/or the guide wire 18, 30 disposed therewithin. In some embodiments, the tensioner is a function of the front gripper 44. The front gripper 44 applies tension to the balloon catheter 10, 22 and/or the guide wire 18 disposed therewithin. The tension causes the guide wire 18, 30 to straighten. In some embodiments, the tension causes the guide wire 18, 30 to assume a substantially linear path between the back griper 40 and the front gripper 44. In some embodiments, the tension causes the guide wire 18, 30 to become coaxial with the longitudinal axis C-C of the cavity 38. In some embodiments, the tensioner is a function added to the front gripper 44, a sliding balloon guide 46, and/or the back gripper 40. In some embodiments, the tensioner is a separate component.

In some embodiments, the guide wire 30 adjusts the position of the bowed inner lumen 24. The bowed inner lumen 24 can become substantially linear due to the tension applied to the guide wire 30. In some embodiments, the tension by the guide wire 30 causes the bowed inner lumen 24 to become coaxial with the longitudinal axis C-C of the cavity 38. The bowed inner lumen 24 can be aligned with the longitudinal axis C-C of the cavity 38 upon applying tension. If the blades 36 were to close on the balloon catheter 22 in this configuration, then the flutes may be even. Once the balloon catheter 22 is fluted, the back griper 40 and the front gripper 44 release the balloon catheter 22. The balloon catheter 22 can be withdrawn from the fluter 34 and the sliding balloon guide 46.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “expanding a balloon” include “instructing the expanding of a balloon.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

What is claimed is:

1. An apparatus for gripping a balloon catheter during fluting comprising:
a base unit;
a back gripper;
a front gripper; and
a plurality of arms coupled to the base unit and arranged in a pattern about a central axis;
wherein the back gripper is removably coupled to a first portion of a balloon catheter and the front gripper is removably coupled to the second portion of the balloon catheter,
wherein the front gripper is configured to straighten an inner lumen of the balloon catheter, wherein the inner lumen aligns with the central axis when straightened.

2. The apparatus of claim 1, wherein the plurality of arms can translate in a radial direction toward and away from the central axis.

3. The apparatus of claim 1, wherein a plurality of blades can be coupled to the plurality of arms such that the plurality of blades can engage the balloon catheter during fluting.

4. The apparatus of claim 1, further comprising the balloon catheter.

5. The apparatus of claim 4, wherein the inner lumen of the balloon catheter is bowed.

6. The apparatus of claim 4, further comprising a guide wire disposed within the inner lumen of the balloon catheter.

7. The apparatus of claim 6, wherein the front gripper is configured to straighten the guide wire to straighten the balloon catheter along an inner lumen of the balloon catheter.

8. The apparatus of claim 1, further a sliding balloon guide.

9. The apparatus of claim 8, wherein the sliding balloon guide is disposed between the back gripper and the front gripper.

10. A method for gripping a balloon catheter during fluting comprising:
providing a device having a base unit, a back gripper, a front gripper;
removably coupling the back gripper to a first portion of a balloon catheter;
removably coupling the front gripper to a second portion of the balloon catheter;
straightening the balloon catheter along an inner lumen of the balloon catheter; and
aligning the inner lumen with a central axis when straightened.

11. The method of claim 10, further comprising providing a plurality of arms coupled to the base unit and arranged in a pattern about a central axis

12. The method of claim 11, further comprising translating the plurality of arms in a radial direction toward and away from the central axis

13. The method of claim 11, fluting the balloon catheter with a plurality of blades coupled to the plurality of arms.

14. The method of claim 10, further comprising providing the balloon catheter.

15. The method of claim 14, wherein the inner lumen of the balloon catheter is bowed.

16. The method of claim 14, further comprising a guide wire disposed within the inner lumen of the balloon catheter.

17. The method of claim 14, wherein straightening the balloon catheter along an inner lumen of the balloon catheter comprises straightening the guide wire to straighten the balloon catheter along an inner lumen of the balloon catheter.

18. The method of claim 10, further comprising releasing the balloon catheter from the back griper and the front gripper.

19. The method of claim 10, further comprising disposing the balloon catheter within a sliding balloon guide.

20. A method for gripping a balloon catheter during fluting comprising:
gripping a first portion of a balloon catheter;
gripping a second portion of the balloon catheter;
straightening the balloon catheter along an inner lumen of the balloon catheter;
aligning the inner lumen with a central axis when straightened;
fluting a balloon of the balloon catheter; and
releasing the gripping of both the first portion of the balloon catheter and the second portion of the balloon catheter.
